(19) United States
(12) Patent Application Publication
(10) Pub. No.: US 2022/0095663 A1
(43) Pub. Date: Mar. 31, 2022

(54) COMPLEX MUSHROOM MYCELIUM COMPOSITION HAVING LIVER FUNCTION-IMPROVING ACTIVITY AND PREPARATION METHOD THEREFOR

(71) Applicant: GIUNCHAN CO., LTD., Cheonan-si, Chungcheongnam-do (KR)

(72) Inventors: Jong Yea PARK, Cheonan-si (KR); Mi Na PARK, Cheonan-si (KR); Hyun Min KIM, Asan-si (KR)

(73) Assignee: GIUNCHAN CO., LTD., Cheonan-si, Chungcheongnam-do (KR)

(21) Appl. No.: 17/426,900

(22) PCT Filed: Dec. 3, 2019
(86) PCT No.: PCT/KR2019/016889

§ 371 Date: Jul. 29, 2021

(30) Foreign Application Priority Data
Jan. 30, 2019 (KR) 10-2019-0012193

(51) Int. Cl. A23L 31/00; C12N 1/14; A23L 33/00; A23L 33/10

(52) U.S. Cl. CPC A23L 31/00; C12N 1/14; A23V 2002/00; A23L 33/10; A23L 33/40

(57) ABSTRACT

The present invention relates to a mushroom mycelium complex composition having a liver function-improving activity and a preparation method therefor. The composition is prepared by collectively inoculating the mycelia of three kinds of mushrooms that are Inonotus obliquus, Ganoderma lucidum, and Phellinus linteus, into a naked barley culture medium to obtain a mushroom mycelium complex and extracting the mushroom mycelium complex to obtain an extract of the mushroom mycelium complex. The composition has an effect of improving liver function.

### TECHNICAL FIELD

The present invention relates to a mushroom mycelium composition for improving liver function. More particularly, the present invention relates to a mushroom mycelium complex composition having a liver function-improving activity and a preparation method therefor, the composition being prepared by collectively inoculating the mycelia of three kinds of mushrooms that are Inonotus obliquus, Ganoderma lucidum, and Phellinus linteus mycelia into a naked barley culture medium to prepare a mushroom mycelium complex and obtaining an extract of the mushroom mycelium complex, the composition providing a liver function-improving effect.

### BACKGROUND ART

The function of a living body is maintained by the balance between the oxidation action and the antioxidation action. Not only the cells of various organs but also skin cells and endothelial cells of blood vessels actively function or suffer hypofunction or aging due to the influence of this oxidation action. In addition, immunological reaction, the development of cancer, anti-cancer activity against cancer cells, and biodefense action against microbial infection are all affected by the oxidation and anti-oxidation actions in the body.

In recent years, patients suffering from so-called adult diseases such as cardiovascular diseases and metabolic disorders, which are caused by environmental pollution and changes in eating patterns, obesity, and an increase in psychological stress in social life, are rapidly increasing. In particular, patients suffering from fatty liver, liver hardening, liver cirrhosis, liver cancer, or hepatitis, which are liver-related diseases, are increasing.

The liver is an organ that is substantially responsible for various functions in the living body, such as the metabolism of fat and the processing of nutritional components, the supply of energy sources, and the like. When liver function is deteriorated due to viral infection or liver cancer, significant abnormality occurs not only in the cardiovascular system but also in nutrient metabolism, resulting in impairment of the biological function.

However, even though many studies related to liver function have been conducted domestically and internationally, no definitive solution has been introduced. The reasons include that the candidate substances for liver function-improving agents, which have been developed so far, exhibit cytotoxicity to liver tissue and there is a large difference between in vitro and in vivo results.

Thus, in the relevant field studies, it is expected that a technique of transforming natural products that cause substantially no side effects on human bodies into a functional food formulation will be more effective rather than developing a novel liver function-improving pharmaceutical formulation. However, about ten active ingredients and substances that have been reported abroad all exhibited hepatotoxicity and side effects, and thus their use is questionable.

One of the causes of deterioration of liver function is when oxidation severely occurs in liver tissue, the function of hepatocytes is reduced, resulting in a total reduction in the biological function. For example, when a large amount of alcohol is ingested, the degradability for alcohol is lowered, resulting in a hangover, the liver function is degraded to be susceptible to microbial infection such as viral infection, and the infection is likely to occur. In addition, when the liver function becomes weakened, the level of cholesterol in blood is increased to cause hyperlipidemia and the like.

In particular, in the case where the function of the liver is deteriorated due to the overall ageing of the body, the person may easily suffer from not only various infectious diseases but also cardiovascular diseases such as heart disease, hyperlipidemia, and hypertension, or metabolic disorders such as diabetes. Therefore, the improvement of liver function is important for the health maintenance of adults, and thus there has been an increasing demand for liver function-improving food formulations that utilize natural products that rarely cause side effects on human bodies.

[symbol]

### RELATED ART LITERATURE

### Patent Literature

Korean Patent Application Publication No. 10-2007-0005950 (Jan. 11, 2007)

### DETAILED DESCRIPTION OF THE INVENTION

### Technical Problem

The present invention has been made in view of the problems occurring in the related art and an objective of the present invention is to provide a mushroom mycelium complex composition having a liver function-improving activity and a preparation method therefor, the mushroom mycelium complex composition being prepared by collectively inoculating mycelia of Inonotus obliquus, Ganoderma lucidum, and Phellinus linteus into a naked barley culture medium and obtaining an extract of the mushroom mycelium complex, the composition thereby providing an effect of improving liver function.

### Solution to Problem

To achieve the objective of the present invention, there is provided a method of preparing a liver function-improving food formulation, the method including: a) water-soaking and dewatering naked barley to form a medium; b) adjusting the pH condition of the medium; c) sterilizing the medium; d) inoculating the medium with an inoculum for a mushroom mycelium complex; e) culturing the inoculum on the medium to obtain a mushroom mycelium complex; f) drying and pulverizing the mushroom mycelium complex; and g) extracting the pulverized mushroom mycelium complex with not water to obtain a liquid extract of the mushroom mycelium complex and concentrating the liquid extract.

In this case, the inoculum may be prepared by: d-1) separately culturing the mycelium of each kind of mushroom after inoculation of the fruit body tissue of each of Inonotus obliquus, Ganoderma lucidum, and Phellinus linteus into a potato dextrose agar (PDA); d-2) collectively inoculating the mycelia of the three kinds of mushrooms cultured in step d-1) into a potato dextrose broth (POB); and d-3) culturing the inoculated mycelia in the POB for 4 to 6 weeks.

In addition, in step c), preferably, the medium is sterilized at 110° C. to 130° C. for 30 minutes to 1 hour and 30 minutes and then cooled.

In addition, in step d), preferably, the inoculum may be inoculated in a concentration of 0.03% to 0.3% by weight per kg of the naked barley culture medium.

In addition, preferably, the liver function-improving food formulation of the present invention may be prepared by the above-described method.

### Effects of Invention

According to the present invention, it is possible to save space for facility installation and produce sanitary products by using a method of growing and producing mycelia of three kinds of mushrooms which are natural substances.

In particular, the mycelium complex of three kinds of mushrooms including Inonotus obliquus, Ganoderma lucidum, and Phellinus linteus has an excellent inhibitory effect on alcohol-induced hepatotoxicity and exhibits not only antioxidation activity but also high anti-inflammatory activity.

### BRIEF DESCRIPTION OF DRAWINGS

FIG. 1 is a flowchart illustrating a method of preparing a liver function-improving food formulation according to a preferred embodiment of the present invention, in which (A) of FIG. 1a and (A′) of FIG. 1b are connected to each other to sequentially advance the process.

FIG. 2 is a graph illustrating the inhibitory effect of the mycelia of three kinds of mushrooms on alcoholic hepatotoxicity.

FIG. 3 is a graph showing the effect of reducing LDH in blood, by the mycelia of three kinds of mushrooms.

FIG. 4 is a graph showing the inhibitory effect of the mycelia of three kinds of mushrooms on alcohol-induced weight loss.

FIG. 5 is a graph showing the inhibitory effect of the mycelia of three kinds of mushrooms on alcohol-induced weight reduction of the liver.

FIG. 6 is a graph showing the effect of mushroom mycelium on the expression of an antioxidant enzyme in liver tissue.

FIG. 7 is a graph showing a comparison of antioxidation activity among mycelia of mushrooms.

FIG. 8 is a graph showing cytotoxicity of mushroom mycelium samples.

FIG. 9 is a graph showing the inhibition of NO production in inflammatory cells by mushroom mycelium samples.

FIG. 10 is a graph showing inhibition of TNF-a production in inflammatory cells by mushroom mycelium samples.

FIG. 11 is a graph showing inhibition of IL-6 production in inflammatory cells by mushroom mycelium samples.

FIG. 12 is a graph showing a comparison of polyphenol content among mushroom mycelia.

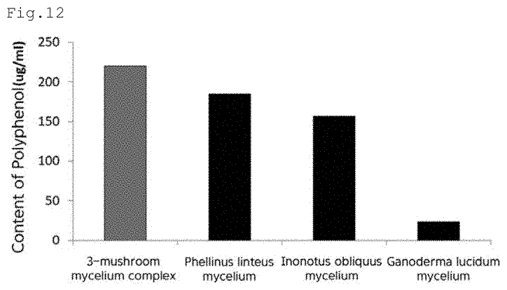

### DESCRIPTION OF EMBODIMENTS

Hereinafter, a mushroom mycelium complex composition having a liver function-improving activity and a preparation method therefor, according to the present invention, will be described in detail with reference to the accompanying drawings.

The liver function improvement targeted by the present invention can be substantially obtained by enhancing the antioxidation activation of hepatocytes. A large amount an antioxidant is contained a plant extract containing a large quantity of a flavonoid-based substance. That is, by developing a liver function-improving food formulation using a plant extract containing a large amount of an antioxidant, it is possible to prevent adult diseases.

Silymarin contained in a milk thistle extract is known as an effective liver function-improving substance. As drugs for treating non-hepatitis viral chronic liver disease, ursodioxycholic acid (UDCA) such as bile acid-modulating drugs, liver extracts, vitamin complexes, liver metabolism promoters such as argin, and detoxifying active substances such as citioxo have been mainly used. However, most of them are synthetic compounds, side effects are still problematic, and the effects thereof are still unsatisfactory.

Mushrooms used in the present invention generally contain a large amount of β-Glucan, which is a component having various physiological activities. The β-Glucan is attracting attention as not only a raw material for new drug development but also a functional food formulation.

Various products obtained by artificially culturing mushrooms and processing the mycelia of the mushrooms have been developed. However, even though there have been various attempts, it was difficult to find a practical food formulation other than simple processed foods, aside from an achievement in which mycelia of mushrooms recognized as functional substances have been produced as drugs.

Therefore, an objective of the present invention is to provide a liver function-improving food formulation derived from a mushroom mycelium complex obtained by collectively culturing the mycelia of three kinds of medicinal mushrooms including Inonotus obliquus, Ganoderma lucidum, and Phellinus linteus on an agricultural produce medium rather than from the mycelium of a single kind of mushroom. Thus, various forms of products can be further developed from the mycelium complex and the competitiveness of the products in the market can be gained through scientific analysis and efficacy demonstration.

[symbol]

### 1. Example

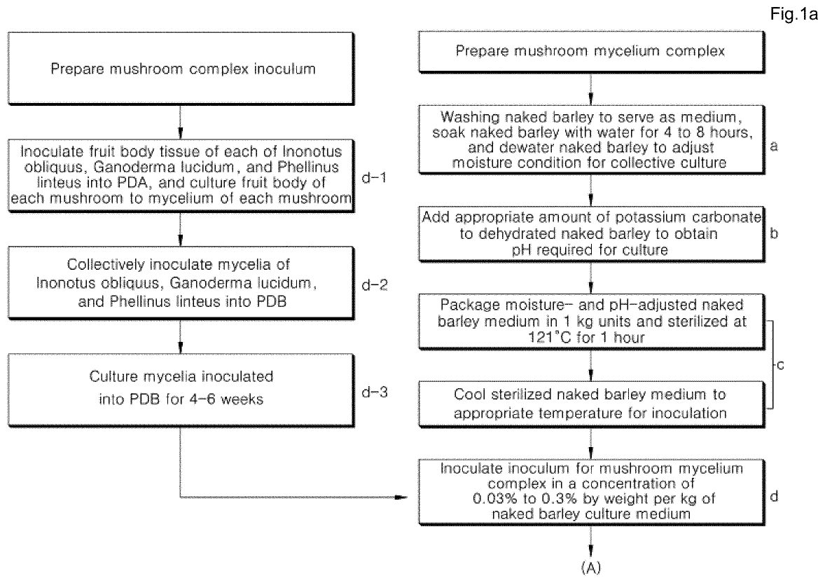

FIG. 1 is a flowchart illustrating a method of preparing a liver function-improving food formulation according to a preferred embodiment of the present invention. The liver function-improving food formulation of the present invention is prepared through a method described below.

a) In the step of water-immersing and dewatering naked barley to form a medium, naked barley was washed, then was immersed in water for 4 to 6 hours so that the naked barley had a water content of 10% to 20% by weight, and preferably 15% by weight, with respect to the total weight thereof. That is, the moisture condition of the medium was adjusted to meet the required level for collective culturing.

b) in the step of adjusting the pH condition of the medium, the pH of the medium was adjusted to 7.0 to 7.2 by adding an appropriate amount of calcium carbonate.

c) In the step of sterilizing the medium, the naked barley culture medium that was adjusted to have a predetermined moisture content and a predetermined pH condition was packaged in 1 kg bags and then sterilized at 110° C. to 130° C. for 30 minutes to 1 hour and 30 minutes, followed by cooling. Preferably, the sterilization was performed at 121° C. for 1 hour and then the medium was cooled to a temperature of 22° C. to 24° C.

d) In the step of inoculating the medium, a mushroom complex inoculum was inoculated into the naked barley culture medium in a concentration of 0.03% to 0.3% by weight per kg of the naked barley culture medium.

In this step, the inoculation was performed by the steps of: d-1) separately culturing the mycelium of each kind of mushrooms that are Inonotus obliquus, Ganoderma lucidum, and Phellinus linteus by inoculating the fruit body tissue of each kind of the mushrooms into a potato dextrose agar (PDA); d-2) collectively inoculating the mycelia (cultured in step d-1)) of the three kinds of mushrooms into a potato dextrose broth (POB); and d-3) culturing the mycelia in the POB for 4 to 6 weeks.

In this step, 100 parts by weight of the medium was inoculated with 30 parts by weight of the fruit body tissue of each kind of mushroom.

e) In the step of culturing in the medium, the mycelia were cultured in the dark for 30 to 40 days.

f) In the step of pulverizing the mycelia of the mushrooms, the mycelia were dried at 57° C. to 60° C. and then pulverized.

g) In the step of hot water extraction and concentration, the mycelia were mixed with distilled water and extracted at 70° C. to 72° C. for 10 to 12 hours. In the step, the weight of the distilled water added for the hot water extraction was 10 times the weight of the pulverized mycelia. After the extraction, the obtained liquid extract was thickened so that the initial volume of the obtained liquid extract was reduced to one fifth. The concentrated liquid extract can be used as a functional food formulation as it is. However, when the liquid extract is transformed into powder, the application range of the mushroom complex mycelia extract can be broadened.

h) The concentrated extract was then dried by heat drying (HD) so that solid extract was obtained. The heat drying was performed 80° C. for 6-8 hours.

[symbol]

### 2.Experimental Methods

1) Cytotoxicity test: Each mycelium sample was diluted into various concentrations, each diluted sample was inoculated into RAW 264.7 cells and cultured for 24 hours, and then cytotoxicity was measured by an MTT method.

2) Mouse alcoholic liver disease model: A 5-week-old male Balb/c mouse was orally administered with 25%-ethanol at a dose of 5 g/kg once a day for a total of 7 days to induce alcoholic liver disease.

3) Administration of sample: Two kinds of test samples including a positive control group were orally administered in an amount of 2 mg/mouse once a day for a total of 9 days from the second previous day before the start of administration of ethanol.

4) Biochemical analysis: GTE (ALT), GOT (AST), and LDH in blood were quantified using serum samples on day 1 after the end of administration of ethanol, and liver tissue was dissolved on day 1 after the end of the administration of ethanol to quantify SOD and catalase in liver tissue.

5) Safety (in vivo test) of the sample: The presence or absence of toxicity for each sample was determined by a hepatotoxicity test including body weight measurement and serological analysis.

6) Anti-inflammatory activity measurement: The anti-inflammatory experiment used a model in which RAW 264.7 macrophages were stimulated with LPS (100 ng/ml) to induce inflammation. Each of the samples was treated for 12 hours before the LPS stimulation which was performed in in various concentrations and was then cultured for 24 hours after the LPs stimulation. Inflammatory factors such as NO, TNF-a, and IL-6 in the cell culture medium were quantified by ELISA after the culturing.

7) Antioxidation activity measurement test: The antioxidation activity of each sample was measured by a DPPH method using 1.1-diphenyl-2-picrylhydrazyl. The positive control group was treated with BHT at a concentration of 50 μg/ml.

8) Statistical significance was examined by Student's two tailed t-test for the groups treated only with ethanol.

[symbol]

### 3. Experimental Results

1) Alcoholic Hepatotoxicity Inhibitory Effect

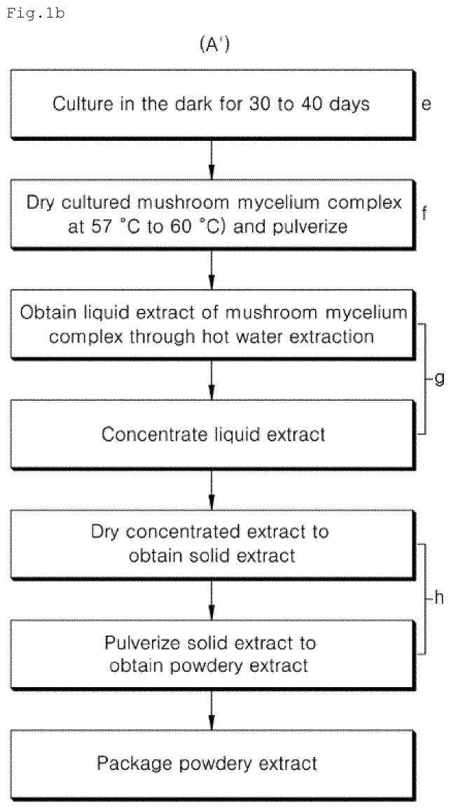

FIG. 2 is a graph showing the effect of inhibiting alcoholic hepatotoxicity by the mycelia of three kinds of mushrooms. GOT and GPT in serum were quantitated to determine the liver disease inhibitory activity of each sample. The results show that the increased blood concentrations of GOT and GPT after the administration of ethanol decreased after the administration of each of the mushroom mycelia. In particular, a mixture of the mycelia of the three kinds of mushrooms showed a higher inhibitory effect than the mycelium of each single kind of mushroom.

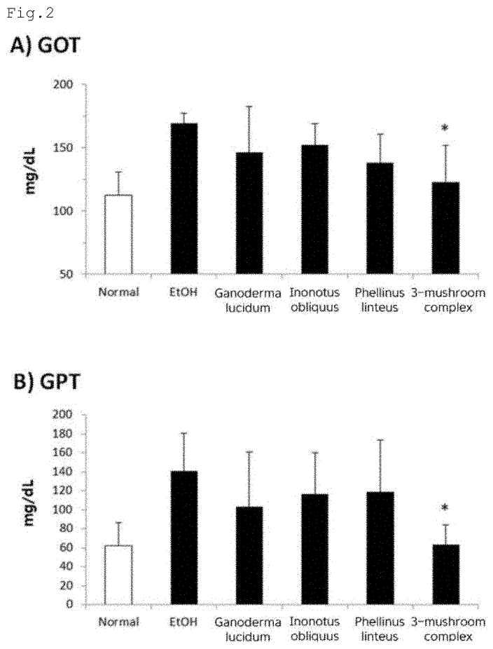

FIG. 3 is a graph showing the effect of reducing LDH in the blood by the mycelia of the three kinds of mushrooms. It is known that the concentration in blood of LDH enzyme in hepatocytes increases due to the damage of hepatocytes during disease development. When observing the inhibitory effect of the mushroom mycelium samples on the increase in the LDH concentration in blood in the liver disease induced by the administration of alcohol, it was confirmed that the inhibitory effect on the increase of the LDH concentration in blood by administration of ethanol was the highest in the case where the mixed mycelia of the three kinds of mushrooms was administered.

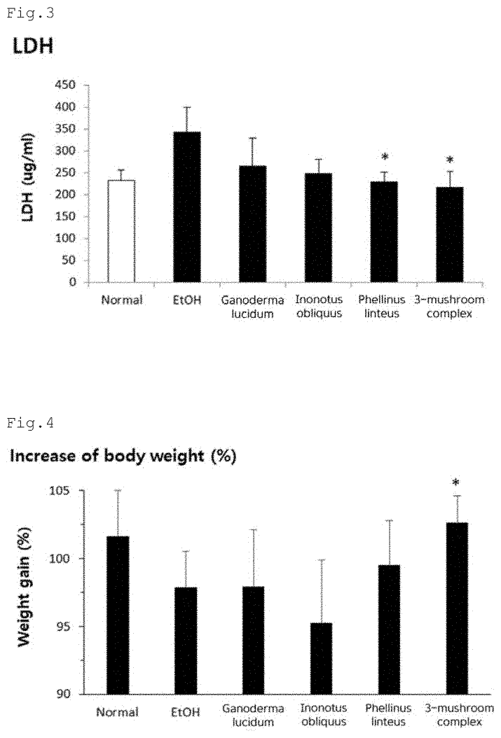

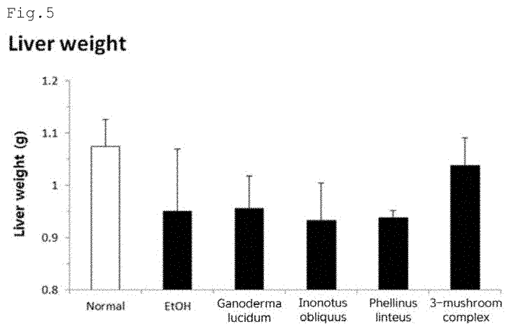

FIG. 4 is a graph showing the inhibitory effect of the mixed mycelia of the three kinds of mushrooms on the weight loss induced by alcohol, and FIG. 5 is a chart showing the inhibitory effect of the mixed mycelia of the three kinds of mushrooms on the weight reduction of liver induced by alcohol. In the case of the inhibitory effect on the decrease of body weight and liver weight induced by alcoholic liver disease, the mixed mycelia showed the highest inhibitory effect.

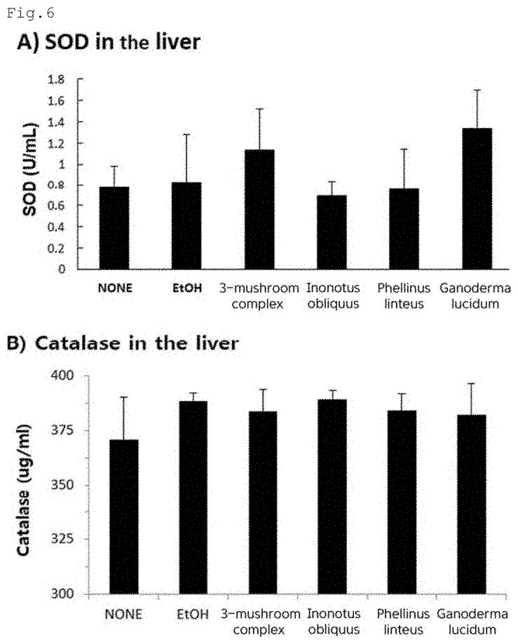

FIG. 6 is a graph showing the effect of the mushroom mycelium on the expression of an antioxidant enzyme in the liver tissue. When measuring the expressions of SOD and catalase which are antioxidant enzymes in the liver tissue, it was shown that the administration of the mixed mycelia of the three kinds of mushrooms exhibited a slight increase in only the SOD expression.

2) Antioxidant Effect of Mushroom Mycelium

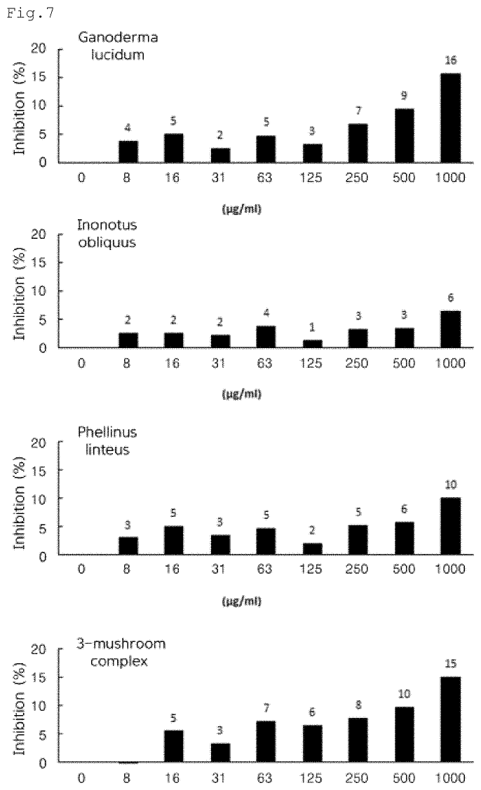

FIG. 7 is a graph showing the comparison of the antioxidation activity among mushroom mycelia, in which the antioxidation activity of each sample was measured by the DPPH method. The graph shows that the mixed mycelia of three kinds of mushrooms exhibited the highest activity. In the case of the positive control group, BHT exhibited 10% inhibition at 50 μg/ml.

3) Anti-Inflammatory Effect of Mushroom Mycelium

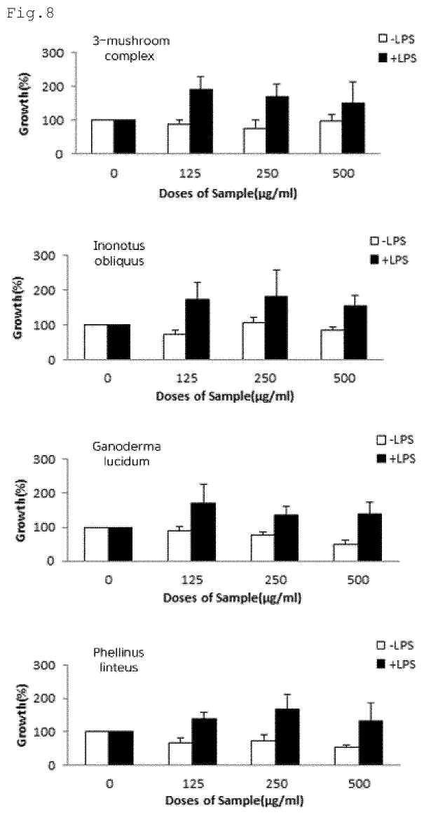

FIG. 8 is a graph showing cytotoxicity of mushroom mycelium samples, and the anti-inflammatory activity was measured using RAW 264.7 cells. First, cytotoxicity of each sample against the cells was measured, and as a result, it was confirmed that all mushroom mycelium samples were safe up to a concentration of 500 μg/ml.

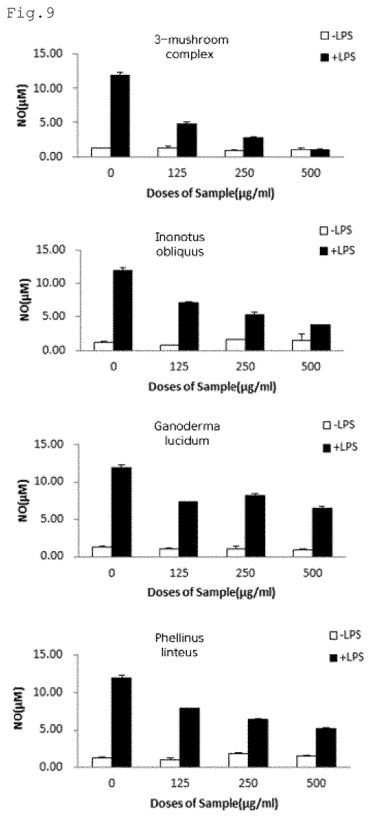

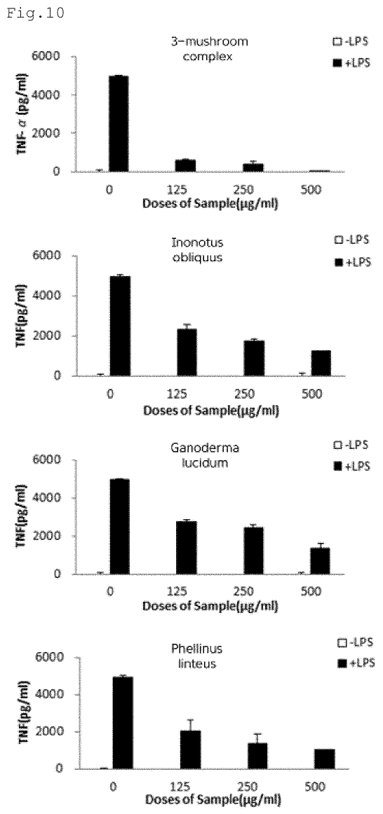

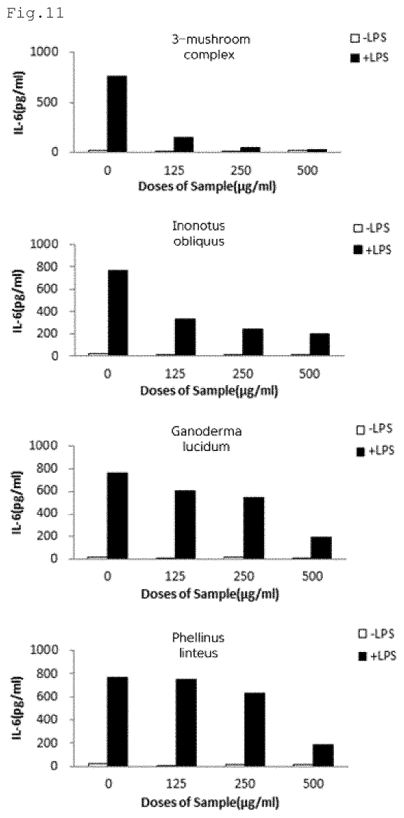

FIG. 9 is a graph showing the NO production inhibition effects of mushroom mycelium samples on inflammatory cells, FIG. 10 is a graph showing the TNF-a production inhibition effects of mushroom mycelium samples on inflammation cells, and FIG. 11 is a graph showing the IL-6 production inhibition effects of the mushroom mycelium samples on inflammatory cells. The test results showed that there was no cytotoxicity. The inhibition on production of NO that is an inflammatory factor was examined. The results showed that the anti-inflammatory effect of the mixed mycelia of the three kinds of mushrooms was highest. In addition, even in terms of the secretion of pro-inflammatory cytokines such as INF-a and IL-6, the mixed mycelia of the three kinds of mushrooms showed the highest inhibitory effect.

4) Quantification of Mushroom Mycelium Polyphenol

FIG. 16 is a graph showing the comparison of polyphenol content of each mushroom mycelium. When the polyphenol content was quantified, it was confirmed that the mixed mycelia of the three kinds of mushrooms had the highest concentration of polyphenol.

[symbol]

4. Results Summary

1) The mixed mycelia of three kinds of mushrooms were found to have an effect of inhibiting alcohol-induced hepatotoxicity, and this effect was confirmed to be higher than that of each group treated with the mycelium of a single kind of mushroom.

2) The effect of inhibiting hepatotoxicity by the mixed mycelia of three kinds of mushrooms were assumed to be related to an increase in the expression of SOD which is an antioxidant enzyme in liver tissue.

3) The mixed mycelia of three kinds of mushrooms were shown to have a higher antioxidation activity than the mycelium of each single kind of mushroom.

4) In addition to the inhibitory effect on hepatotoxicity, the mixed mycelia of three kinds of mushrooms showed a high anti-inflammatory activity.

5) The hot water extract of the mixed mycelia of three kinds of mushrooms was confirmed to have a high anti-inflammatory activity.

6) The extract of the mixed mycelia of three kinds of mushrooms was confirmed to contain a larger amount of polyphenol than the extract of the mycelium of each single kind of mushroom.

[symbol]

It is to be understood that the right of the present invention is not limited to the examples described above, but is defined as set forth in the claims, and that various modifications and adaptations may be made by those skilled in the art without departing from the scope of the claims.

1. A method of preparing a liver function-improving food formulation, the method comprising:
a) water-soaking and dewatering naked barley to obtain a naked barley medium;
b) adjusting the pH condition of the medium;
c) sterilizing the medium;
d) inoculating the medium with a mushroom complex inoculum;
e) culturing the inoculum on the medium to obtain a mushroom mycelium complex;
f) drying and pulverizing the mushroom mycelium complex; and
g) obtaining a liquid extract of the mushroom mycelium complex through hot water extraction and concentrating the liquid extract of the mycelium complex.

2. The method according to claim 1, wherein the inoculum is prepared through the steps of:
d-1) separately culturing the mycelium of each of three kinds of mushrooms that are Inonotus obliquus, Ganoderma lucidum, and Phellinus linteus after separately inoculating the fruit body tissue of each kind of mushroom into a potato dextrose agar (PDA);
d-2) collectively inoculating the mycelia of the three kinds of mushrooms cultured in the step d-1) into a potato dextrose broth (POB); and
d-3) culturing the collective mycelia of the three kinds of mushrooms in the POB for 4 to 6 weeks.

3. The method according to claim 1, wherein in step c), the medium is sterilized at 110° C. to 130° C. for 30 minutes to 1 hour and 30 minutes and then cooled.

4. The method according to claim 1, wherein in step d), the inoculum is inoculated in a concentration of 0.03% to 0.3% by weight with respect to 1 kg of the naked barley culture medium.

5. A liver function-improving food formulation prepared by the method of claim 1.
